(19) United States
(12) Patent Application Publication
(10) Pub. No.: US 2007/0208228 A1
(43) Pub. Date: Sep. 6, 2007

(54) Surgical retractors and methods of minimally invasive surgery

(76) Inventors: Nicholas Pavento, Walpole, MA (US); Anne Drzyzga, Taunton, MA (US); Connie Marchek, Foxboro, MA (US); William Frasier, New Bedford, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/365,485

(22) Filed: Mar. 1, 2006

(51) Int. Cl. A61B 1/32

(52) U.S. Cl. 600/233

(57) ABSTRACT

A retractor includes a frame having a linear side having a first end and a second end and a curvate side connected at a first end to the first end of the linear side and at a second end to the second end of the linear side, a first retractor blade connected to the linear side of the frame, and a second retractor blade connected to the curvate side of the frame.

### BACKGROUND

In surgical procedures, it is important to minimize trauma to the patient and damage to tissue to facilitate patient recovery. One way to accomplish this is to minimize the size of the incision for the surgical procedure and minimize the cutting of tissue to access the target anatomy. A number of retractors are available that are designed to expand a small surgical incision and provide access to a surgical site. Such retractors typically include two or more retractor blades that separate to expand the incision and create an access channel through which to conduct the surgical procedure. One problem with such retractors is that the retractors can be cumbersome, difficult to operate, and time consuming to use.

### SUMMARY

Disclosed herein are retractors and methods of minimally invasive surgery that minimize tissue trauma, facilitate access to a surgical site, such as proximate spinal anatomy, and are less cumbersome and reduce fiddle factor compared with traditional access devices. In one exemplary embodiment, a retractor may comprise a frame having a linear side having a first end and a second end and a curvate side connected at a first end to the first end of the linear side and at a second end to the second end of the linear side, a first retractor blade connected to the linear side of the frame, and a second retractor blade connected to the curvate side of the frame.

In another exemplary embodiment, a retractor may comprise a frame having a linear side having a first end and a second end and a curvate side connected at a first end to the first end of the linear side and at a second end to the second end of the linear side, a first retractor blade connected to the linear side of the frame, and a second retractor blade connected to the curvate side of the frame. The second retractor blade may be rotatable about the curvate side of the frame and the first retractor blade may be rotatable about the linear side of the frame. The retractor may have an insertion configuration in which the distal end of the first retractor blade and the distal end of the second retractor blade are rotated into proximity to one another and a retracted configuration in which the distal end of the first retractor blade and the distal end of the second retractor blade are rotated away from one another.

A method of providing minimally invasive access to spinal anatomy may comprise positioning a retractor in an insertion configuration by rotating a distal end of a first blade of a retractor into proximity to a distal end of a second blade of a retractor. The first blade of the retractor may be connected to a linear side of a frame of the retractor and the second blade may be connected to a curvate side of the frame of the retractor. The method may include making an incision, inserting the distal end of the first blade and the distal end of the second blade of the retractor through the incision with the retractor in the insertion configuration, advancing the distal end of the first blade and the distal end of the second blade into proximity to the spinal anatomy with the retractor in the insertion configuration, and adjusting at least one of the first blade and the second blade relative to the other blade to provide an access channel between the skin and the spinal anatomy.

### BRIEF DESCRIPTION OF THE FIGURES

These and other features and advantages of the devices and methods disclosed herein will be more fully understood by reference to the following detailed description in conjunction with the attached drawings in which like reference numerals refer to like elements through the different views. The drawings illustrate principles of the devices and methods disclosed herein and, although not to scale, show relative dimensions.

FIG. 1 is a perspective view of an exemplary retractor, illustrating the retractor in a first, insertion configuration;

FIG. 2 is a side view of the retractor of FIG. 1, illustrating the retractor in a first, insertion configuration;

FIG. 3 is a side view of the retractor of FIG. 1, illustrating the retractor in a first, insertion configuration and illustrating the secondary blades of the first retractor blade and the second retractor blade advanced relative to a respective primary blade of the first retractor blade and the second retractor blade;

FIG. 4 is a perspective view of the retractor of FIG. 1, illustrating the retractor in a second, retracted configuration;

FIG. 5 is a side view of the retractor of FIG. 1, illustrating the retractor in a second, retracted configuration;

FIG. 6 is a partially sectioned perspective view of the second retractor blade of the retractor of FIG. 1;

FIGS. 7A-B are side views of the second retractor blade of the retractor of FIG. 1, illustrating a mechanism for connecting the second retractor blade to the frame of the retractor;

FIG. 8 is a partially sectioned side view of the second retractor blade of the retractor of FIG. 1, illustrating a mechanism for adjustment of the second retractor blade relative to the frame of the retractor;

FIG. 9 is a perspective view of another exemplary retractor, illustrating the retractor in a closed, insertion configuration; and

FIGS. 10A-D are perspective (FIGS. 10A & 10C) and side views (FIGS. 10B and 10D) of an alternative embodiment of a retractor, illustrating rotation of one segment of the frame of the retractor relative to another segment of the frame of the retractor.

### DETAIL DESCRIPTION OF EXEMPLARY EMBODIMENTS

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

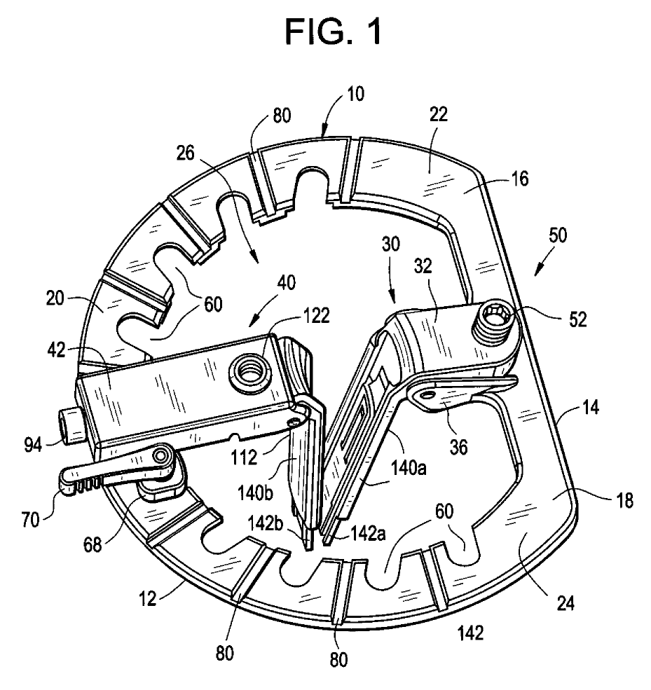

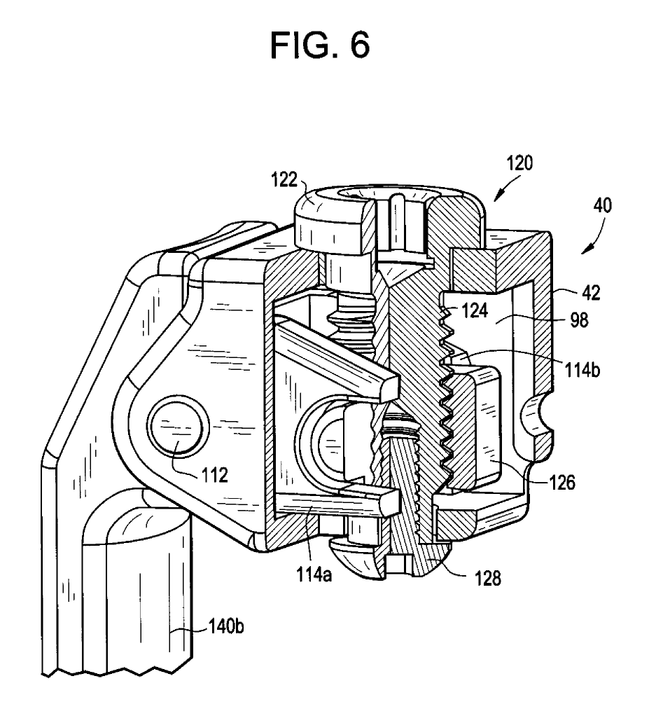

FIGS. 1-8 illustrate an exemplary embodiment of a retractor 10 suitable for providing a selectively expandable access channel through which a surgical procedure may be performed on target anatomy. The exemplary retractor or retractor 10 is particularly suited for minimally invasive spine surgery and, to this end, may be inserted through a relatively small incision to provide a selectively expandable access channel from the skin to the target spinal anatomy. The exemplary retractor 10 includes a frame 12 having a linear side 14 having a first end 16 and a second end 18 and a curvate side 20 connected at a first end 22 to the first end 16 of the linear side 14 and at a second end 24 to the second end 18 of the linear side 14. The exemplary retractor 10 may include a first retractor blade 30 connected to the linear side 14 of the frame 12 and a second retractor blade 40 connected to the curvate side 20 of the frame 12.

The frame 12 of the exemplary retractor 10 is approximately D-shaped including a curvate side 20 having an approximately constant radius along the length of curvate side 20 that connects at both ends 22 and 24 to the ends 16 and 18 of the linear side 14. A D-shaped frame 12 provides a central opening 26 through which a number of retractor blades may be positioned to form and selectively expand an access channel from the frame to the target anatomy. A D-shaped frame 12 is particularly suited for use in providing access to the lumbar spine through a posterior approach. For example, the linear side 14 of the frame 12 may be positioned medially with respect to the spine and may be oriented parallel to the spine, e.g., the linear side 14 may be oriented in the cephalad-caudal direction. In such an orientation, the curvate side 20 of the frame 12 is positioned lateral to the linear side 14 and the spine. The length L of the linear side 14 of the frame 12 may be selected to permit access to one or more levels of the spine through the frame 12. In one exemplary embodiment, the length L is approximately 40 mm to approximately 120 mm, and is preferably approximately 80 mm, to permit access to multiple levels of the lumbar spine through the frame 12 of the retractor 10. The width W of the central opening 26 is selected to provide access to the spinal anatomy in the medial-lateral direction. In one embodiment particularly suited for posterior access to the lumbar spine, the width W of the central opening 26 is approximately 30 mm to approximately 110 mm, and is preferably approximately 72 mm.

In alternative embodiments, the frame 12 may have different shapes and sizes depending on the selected approach and the target anatomy. For example, the curvate side 20 may include a plurality of curvate segments having differing radii and/or may include one or more linear segments.

In the retractor 10 of the exemplary embodiment, the first retractor blade 30 may be fixed to the linear side 14 of the frame 12 to inhibit movement of the first retractor blade 30 along the longitudinal axis 28 of the linear side 14 and to inhibit movement of the first retractor blade 30 in a direction transverse to the longitudinal axis 28 of the linear side 14. By fixing the first retractor blade 30 to the linear side 14 of the frame 12 in this manner, the access channel formed by the retractor blades of the retractor 10 may be expanded proximate frame 12 primarily by adjustment of the second retractor blade 40 or additional retractor blades other than the first retractor blade 30, thereby simplifying expansion of the access channel. The first retractor blade 30 may be centrally located along the length L of the linear side 14 as in the illustrated embodiment or may be positioned at other locations along the length of the linear side 14. In the illustrated embodiment, a single fixed (in translation) retractor blade 30 is provided. In alternative embodiments, additional retractor blades may be connected to the linear side 14.

In the exemplary embodiment, the first retractor blade 30 may be rotatable relative to the linear side 14 of the frame 12. In this manner the first retractor blade 30 of the illustrated embodiment may be fixed in translation relative to the linear side 14, e.g., fixed in a direction transverse to the longitudinal axis 28 of the linear side 14, and may rotate or pivot relative to the linear side 14 of the frame 12. For example, the proximal end 32 of the first retractor blade 30 may rotate about a rotation axis defined by a shaft 34 received by the proximal end 32 of the first retractor blade 30, as indicated by arrow A. The shaft 34 may be connected at one end to a first flange 36 and at a second end to a second flange 38. The first flange 36 and the second flange 38 may extend from the top surface of the linear side 14 of the frame 12 and may be spaced apart to receive the proximal end 32 of the first retractor blade 30 therebetween.

The first retractor blade 30 may include a blade adjustment mechanism 50 for selectively adjusting the rotational position of the first retractor blade 30. In the exemplary embodiment, for example, the blade adjustment mechanism 50 may comprise a set screw 52 engaged to a portion of the first retractor blade 30. The set screw 52, for example, may have external threads that engage internal threads of a hole provided in the proximal end 32 of the first retractor blade 30. The distal end 54 of the set screw 52 may contact the frame 12, for example, on the linear side 14 of the frame 12. Rotation of the set screw 52 about the axis of the set screw 52 advances or retracts the proximal end 32 of the first retractor blade 30 relative to the set screw 52 causing the first retractor blade 30 to rotate about the rotation axis of the shaft 34. One skilled in the art will appreciate that other blade adjustment mechanisms may be used to adjust the rotational position of the first retractor blade 30 including, for example, one or more levers, gears, springs, ratchets or the like.

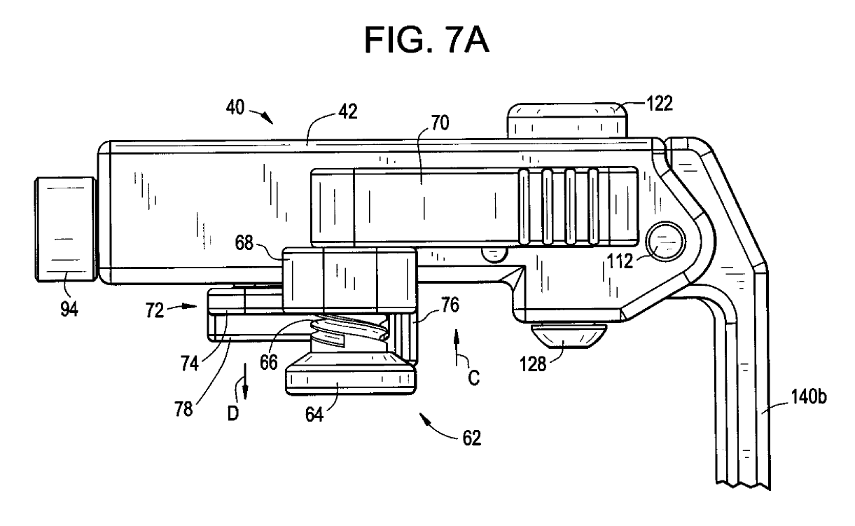

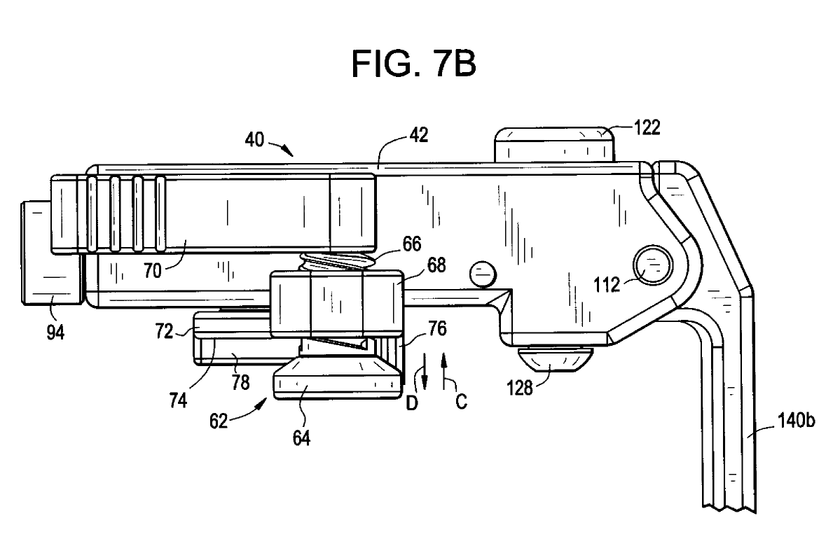

In certain embodiments, the second retractor blade 40 may be connected at a plurality of locations along the length of the curvate side 20 of the frame 12. For example, the curvate side 20 of the frame 12 may be configured to receive one or more retractor blades, such as the second retractor blade 40, along the length of the curvate side 20 of the frame 12. In the exemplary embodiment, for example, the curvate side 20 of the frame 12 includes a plurality of approximately U-shaped cut-outs 60 spaced apart along the length of the curvate side 20 of the frame 12. The U-shaped cut-outs 60 may receive a connection mechanism of a retractor blade to facilitate selective connection of the retractor blade to the curvate side 20 of the frame 12. Referring to FIGS. 7A-B, for example, the second retractor blade 40 may include a connection mechanism comprising a screw 62 having a conical seat 64 and a threaded shank 66 that is received by a threaded collar 68 connected to a proximal end 42 of the second retractor blade 40. A lever 70 (or a handle or other gripping structure) may be connected to the shank 66 of the screw 62 to facilitate rotation of the screw 62 relative to the collar 68. Rotation of the screw 62 by the lever 70 causes the conical seat 64 of the screw 70 to advance toward the collar 68 (arrow C) or move away from the collar 68 (arrow D). The shank 66 of the screw 62 may be sized and shaped to seat within a U-shaped cut-out of the frame 12. The lever 70 and screw 62 may be adjusted between a first, release position in which the conical seat 64 is displaced from the collar 68 and the retractor blade may be removed from the frame 12, illustrated in FIG. 7A, and a second, capture position in which the conical seat 64 is advanced toward the collar, illustrated in FIG. 7B, to capture a portion of the frame 12 between the conical seat 64 and the collar 68 and thereby connect the retractor blade to the frame 12.

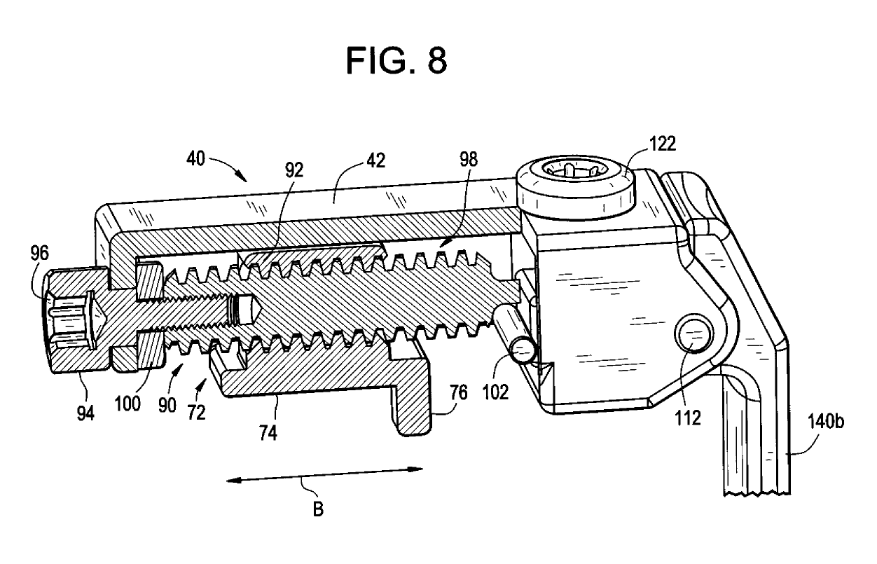

In certain exemplary embodiments, the second retractor blade 40 and other retractor blades may include a structure for aligning the retractor blade relative to the frame 12 of the retractor 10. For example, the second retractor blade 40 of the exemplary embodiment includes an approximately L-shaped (in cross section) alignment member 72 connected to a bottom surface of the proximal end 42 of the second retractor blade 40. Referring to FIGS. 7A, 7B, and 8, the L-shaped alignment member 72 includes a generally planar base 74 for contacting the top surface of the frame 12 and an edge 76 extending transverse to the base 74 for engaging the radially inner edge of the curvate side 20 of the frame 12. The alignment member 72 may include a ridge 78 extending from the base 74 and oriented transverse to the edge 76 of the alignment member 72. The ridge 78 of the alignment member 72 is sized to seat within one of a plurality of radially oriented grooves 80 spaced about the length of the curvate side 20. The ridge 78 and the edge 76 of the alignment member 72 cooperate to maintain alignment of the retractor blade during adjustment of the retractor blade relative to the frame 12.

In the exemplary embodiment, the second retractor blade 40 may be adjustable relative to the curvate side 20 of the retractor frame. For example, the second retractor blade 40 may be adjustable in a direction transverse to an axis defined by the curve of the curvate side 20 of the frame 12, as indicated by arrow B, to selectively expand the access channel created by the retractor blades of the retractor 10. The second retractor blade 40 may include a translational adjustment mechanism to selective adjust the position of the retractor blade 40 relative to the frame 12. Referring to FIG. 8, in the exemplary embodiment, for example, the translational alignment mechanism of the second retractor blade 40 comprises a elongated screw 90 that engages an internally threaded collar 92 connected to the alignment member 72. The elongated screw 90 includes a handle 94 having a tool receiving recess 96 to facilitate rotation of the screw 90 relative to the collar 92 and the frame 12 when connected to the collar 92 through alignment member 72. The screw 90 is positioned within a housing 98 provided in the proximal end 42 of the second retractor blade 40. A portion of the wall of the housing 98 is secured between a washer 100 and the handle 94 such that the handle 90 and screw may rotate relative to the housing 98 and the housing 98 translates with the screw 90 in a direction parallel to the longitudinal axis of the screw 90. A stabilizing bar 102 may be provided across the housing 98 to engage an end of the screw 90 opposite the handle 94 to stabilize the screw 90 during rotation. Rotation of the handle 94 about the longitudinal axis of the screw 90 causes the screw 90 to rotate relative to the collar 92 and, thus, causes the screw 90 and the second retractor blade 40 to move in a direction parallel to the longitudinal axis of the screw 90.

In alternative retractor blade embodiments other translational adjustment mechanisms may be utilized including, for example, gears, ratchets, springs, and/or other adjustment mechanisms.

In certain exemplary embodiments, the second retractor blade 40 or other retractor blades connected to the frame 12 may be rotatable relative to the curvate side 20 of the frame 12. In the illustrated embodiment, for example, the tissue engaging blade of the second retractor blade 40, for example the primary blade 140 and the secondary blade 142b discussed below, is rotatably connected to the housing 98 of second retractor blade 40 by a rotation shaft 112, which defines a rotation axis about which the primary blade 140b, 142b rotates. The rotation shaft 112 spans the housing 98 proximate the proximal end of the primary blade 140b and is rotatably connected to a pair of spaced apart flanges 114A, 114B extending from the proximal end of the primary blade 140b. A rotational adjustment mechanism may be provided to adjust the rotational position of the primary blade 140b of the second retractor blade 40 relative to the frame 12. In the illustrated embodiment, for example, the rotational adjustment mechanism may comprise a screw 120 having a head 122 and a threaded shank 124 received within an internally threaded nut 126 that is connected to the flanges 114A, 114B. The threaded shank 124 of the screw 120 is maintained within the housing 98 of the second retractor blade 40 by a secondary screw 128. Rotation of the screw 120 about the axis of the screw 120 causes the nut 126, and the flanges 114A, 114B, to move along the axis of the screw 120 and, thus, causes the primary blade 140 to rotate about the rotation axis defined by the rotation shaft 112.

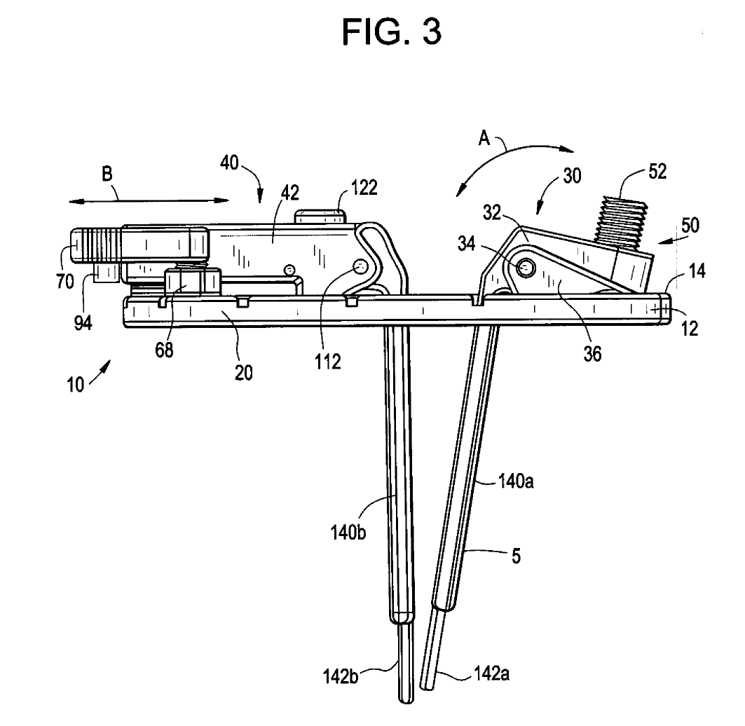

One or more of the blades of the retractor may have an adjustable length, e.g. the blade may telescope to selectively adjust the length of the blade. Referring to FIGS. 1 and 3, for example, one or more of the blades may include a primary blade 140 and an adjustable blade 142 that is operatively coupled to the primary blade and is adjustable relative to the primary blade 140 along the length of the primary blade 140. In the exemplary embodiment, the first retractor blade 30 and the second retractor blade 40 may be adjustable in length and include a respective primary blade 140a,b and a respective adjustable blade 142a,b. Exemplary tissue engaging blades having an adjustable length are disclosed in U.S. Patent Application Publication No. 2005-0137461 A1, which is incorporated herein by reference. The telescoping blades may include a mechanism for selectively adjusting the position of the adjustable blade 142 relative to the primary blade 140. For example, the primary blade 140 may include a plurality of teeth extending along the longitudinal axis of the primary blade 140 and the adjustable blade 142 may include a flexible tab for engaging the teeth of the primary blade 140. The retractor may be inserted through an incision with the adjustable blades 142 in place, as in the case of the exemplary retractor 10 illustrated in FIGS. 1-8. Alternatively, the retractor may be inserted through an incision without the adjustable blades in place. In such embodiments, the retractor may be inserted with the primary blades and one or more adjustable blades may be added after insertion.

The components of the retractors disclosed herein may be manufactured from any biocompatible material including metals, such as stainless steel or titanium, polymers, or composite materials. The components, such as the blades and the frame, may be constructed from the same or different materials.

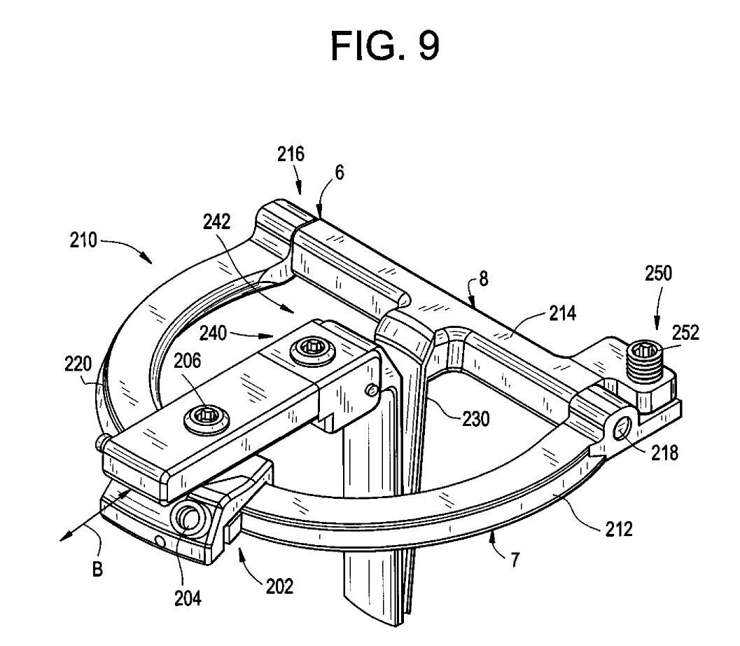

FIG. 9 illustrates another exemplary embodiment of a retractor 210 including a frame 212 having a linear side 214 and a curvate side 220, a first retractor blade 230 connected to the linear side 214 of the frame 212, and a second retractor blade 240 connected to the curvate side 220 of the frame 212. In the exemplary embodiment, the first retractor blade 230 is fixed to the linear side 214 of the frame 212 and the linear side 214 of the frame 212 is rotatably connected to the curvate side 220 of the frame 212. For example, the first end 216 of the linear side 214 and the second end 218 of the linear side 214 each may terminate in a shaft that is received in a cylindrical opening provided at a respective end of the curvate side 220. The exemplary retractor 210 may include a blade adjustment mechanism 250 for selectively adjusting the rotational position of the linear side 214, and, thus, the first retractor blade 230, relative to the curvate side 220 of the frame 212. In the exemplary embodiment, for example, the blade adjustment mechanism 250 may comprise a set screw 252 analogous in construction and operation to the set screw 52 described above in connection with the exemplary retractor 10.

In the exemplary embodiment, the curvate side 220 of the frame 212 and the second retractor blade 240 are configured to permit infinite adjustment of the second retractor blade 240 along the length of the curvate side 220. For example, the second retractor blade may include a connection mechanism for connecting the retractor blade to curvate side 220 of the frame 212 at any position along the length of the curvate side 220. The connection mechanism may comprise a clamp 202 that may slide along the length of the curvate side 220 and may be selectively fixed at a desired location by rotation of a screw 204 directly or indirectly into contact with the curvate side 220 of the frame 212.

The second retractor blade 240 may be adjustable relative to the curvate side 220 of the retractor frame. For example, the second retractor blade 240 may be adjustable in a direction transverse to an axis defined by the curve of the curvate side 220 of the frame 212 to selectively expand the access channel created by the retractor blades of the retractor 210. The second retractor blade 240 may include a translational adjustment mechanism to selective adjust the position of the retractor blade 240 relative to the frame 212. In the exemplary embodiment, for example, the translational adjustment mechanism comprise a gear that engages a rack positioned in the housing at the proximal end 242 of the second retractor blade 240 and may be oriented transverse to the curvate side 220 of the frame 212 when the retractor blade is connected to the frame 212. Rotation of the gear by a screw 206 causes the rack, and, thus, the second retractor blade, to move relative to the frame 212 in a direction parallel to the axis of the rack as indicated by arrow B in FIG. 9.

The second retractor blade 240 or other retractor blades connected to the frame 212 may be rotatable relative to the curvate side 220 of the frame 212 in a manner analogous to the second retractor blade 40 of the exemplary retractor 10 described above.

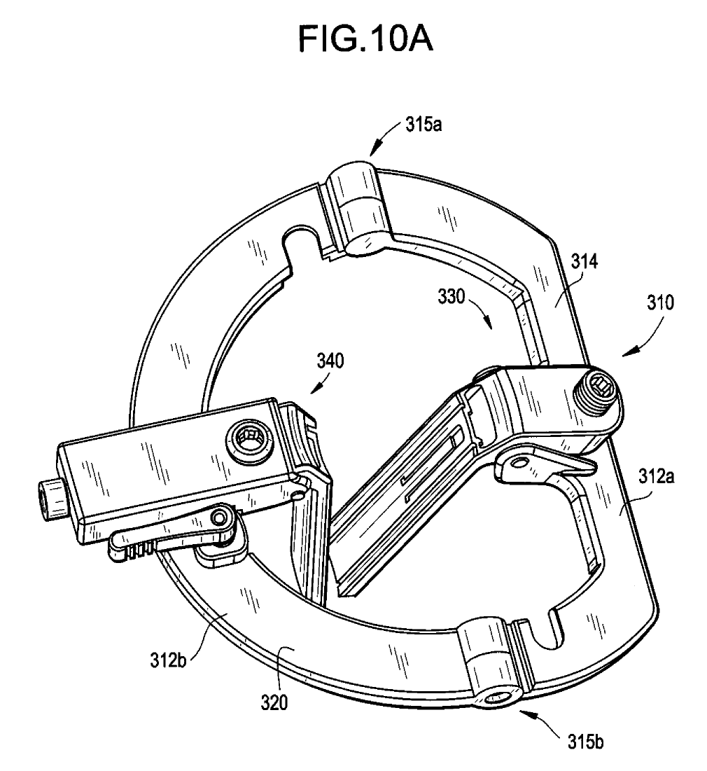

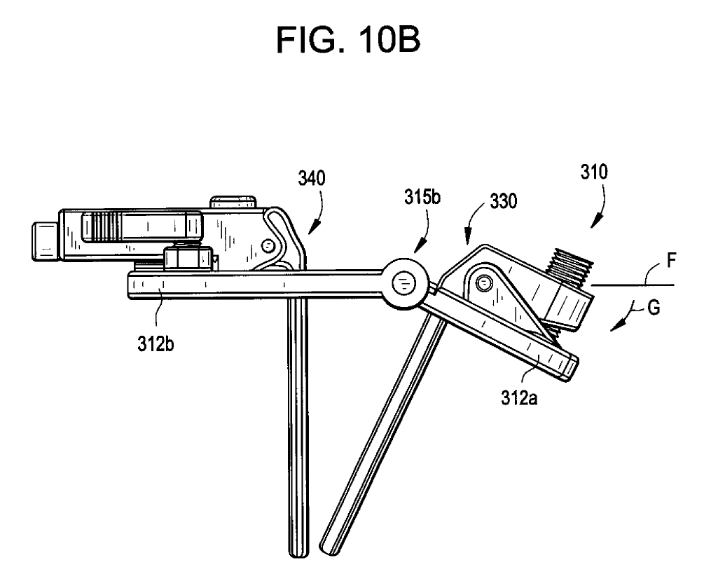

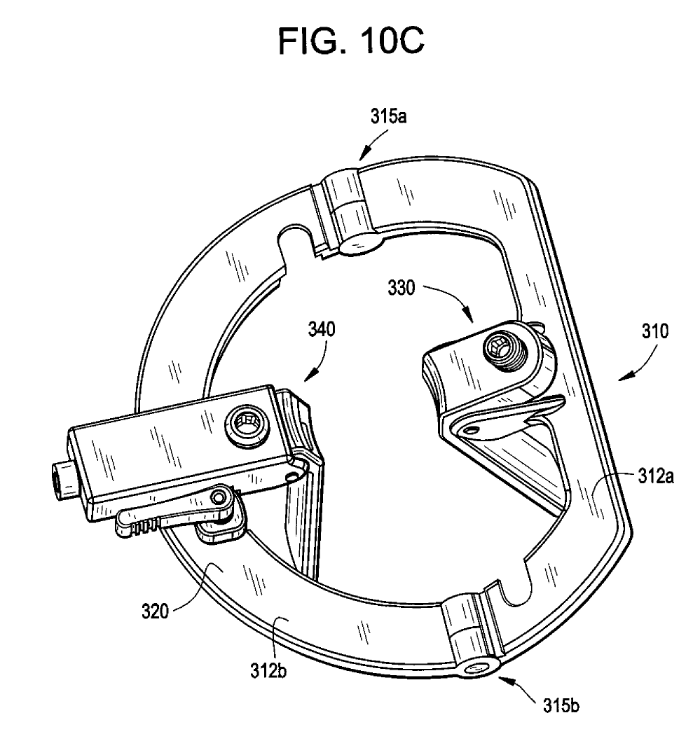

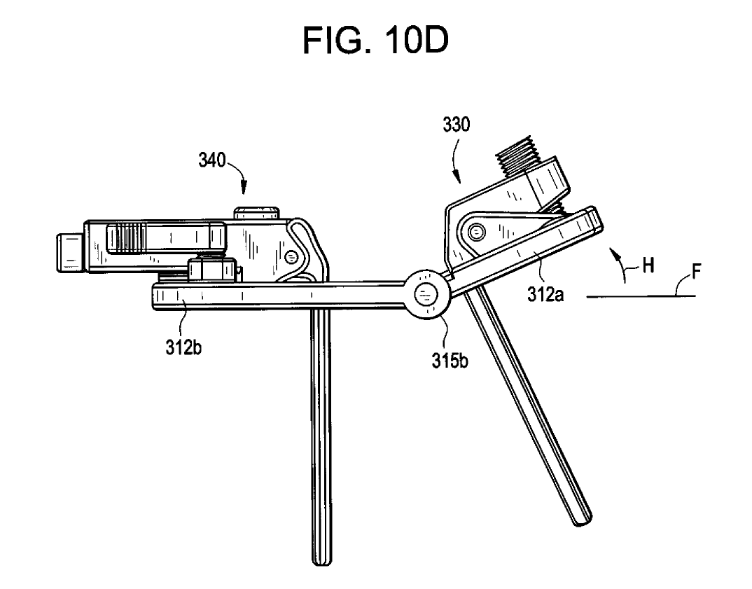

FIGS. 10A-10D illustrated another exemplary embodiment of a retractor 310 a frame 312, a first retractor blade 330 connected to the linear side 314 of the frame 312, and a second retractor blade 340 connected to the curvate side 320 of the frame 312. In the exemplary embodiment, a first segment 312a of the frame 312 may be rotationally adjustable relative to another segment 312b of the frame 312 to provide the ability to conform the frame 312 to the contours of the patient's skin to facilitate positioning of the frame 312 against the skin of the patient. The exemplary frame 312 may include a pair of hinges 315a, 315b that allow the first segment 312a and the second segment 312b to rotate about one another. The location of the hinges 315a, 315b may be varied depending on the desired location of the axis of rotation for the segments 312a, 312b of the frame 312. In alternative embodiments, the frame 312 may include multiple segments, e.g., three, four, or five segments, that are rotatable relative to other segments of the frame.

In the exemplary embodiment, the first segment 312a may be rotated upwards from a neutral plane in which the first segment 312a and the second segment 312b are approximately parallel to one another, indicated by line F, as indicated by arrow H in FIG. 10D. The first segment 312a may be rotated downwards from the neutral plane, indicated by line F, as indicated by arrow G in FIG. 10B.

The frame 312 may have any shape depending on the approach and target anatomy, for example. In the illustrated exemplary embodiment, the frame 312 has a linear side 314 and a curvate side 320. In other exemplary embodiments, the frame 310 may be, for example, circular, oval, elliptical, or rectilinear in shape.

The first retractor blade 330 or other retractor blades connected to the frame 312 may be constructed and may be operable in a manner analogous to the first retractor blade 40 of the exemplary retractor 10 described above or the first retractor blade 230 and frame 212 of the exemplary retractor 210 described above.

The second retractor blade 340 or other retractor blades connected to the frame 312 may be constructed and may be operable in a manner analogous to the second retractor blade 40 of the exemplary retractor 10 described above or the second retractor blade 240 of the exemplary retractor 210 described above.

An exemplary method of providing minimally invasive access to spinal anatomy employing a retractor disclosed herein may include making a skin incision for insertion of the retractor. The incision initially may be less than the diameter of the retractor in a first, insertion configuration, described below. The incision may be expanded to accommodate the retractor by dilation, for example, by placing one or more dilators through the incision to expand the incision in a stepwise manner. The dilators may be employed to separate or dissect the underlying tissue to the target spinal anatomy. Alternatively, the surgeon may employ his finger or the retractor to dissect the underlying tissue and to expand the initial incision. Alternatively, the blades of the retractor may be employed to separate or dissect the underlying tissue to the target spinal anatomy without the use of a dilator.

A retractor may be selected and configured to create an access channel to the target spinal anatomy. For example, the exemplary retractor 10 may be selected and configured by determining the number of retractor blades to be initially inserted through the incision. In one exemplary method described below, the first retractor blade 30 and the second retractor blade 40 are selected for initial insertion through the incision. In alternative methods, only the first retractor blade 30 may be selected for initial insertion or additional retractor blades beyond the first and second retractor blades (e.g., a third and/or fourth retractor blade) may be selected for initial insertion.

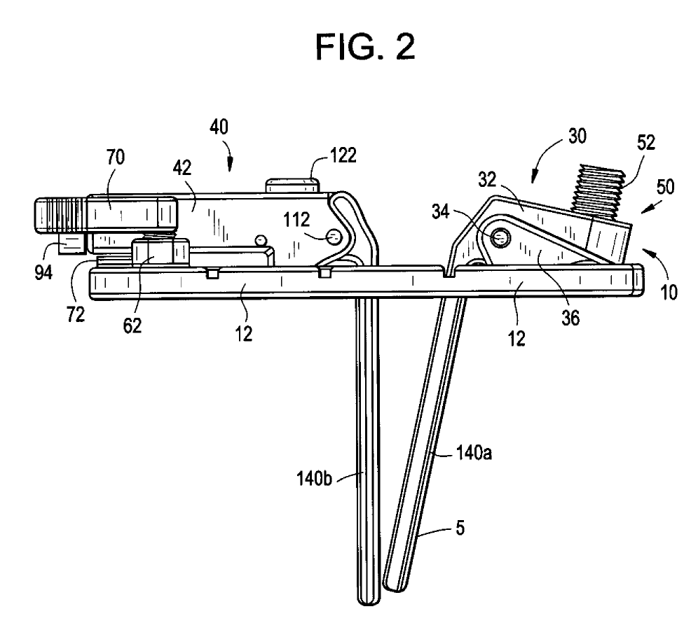

The retractor 10 may be configured for initial insertion by positioning the first retractor blade 30 and the second retractor blade 40 to a first, insertion configuration, in which the first retractor blade 30 and the second retractor blade 40 are adjusted into proximity to one another. For example, the second retractor blade 40 may be adjusted in a direction transverse to the axis of the curvate side 20 of the frame 12 toward the first retractor blade 30 and the distal end of the first retractor blade 30 and the distal end of the second retractor blade 40 may be rotated into proximity to one another, as illustrated in FIGS. 1 and 2.

The first and second retractor blades 30, 40 of the retractor 10 may be inserted through the incision and the distal ends of the blades may be advanced into proximity to the spinal anatomy. The first and second retractor blades 30, 40 are preferably advanced in the first, insertion position, in which the blades are proximate to each other. Once advanced to the target spinal anatomy, the first and second retractor blades 30, 40 form an access channel between the frame 12, which is located at the surface of the skin, and the distal ends of the blades proximate the target spinal anatomy.

In the case of a posterior approach to the spine, the frame 12 of the exemplary retractor 10 may be oriented such that the linear side 14 of the frame 12 is positioned medially with respect to the spine and the linear side 14 is oriented parallel to the spine. In such an orientation, the curvate side 20 is positioned laterally with respect to the spine and the linear side 14 of the frame 12.

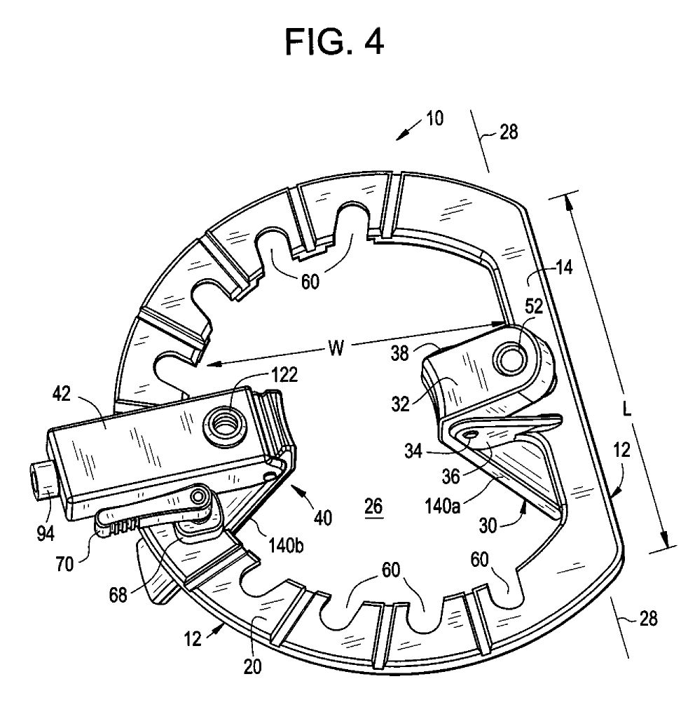

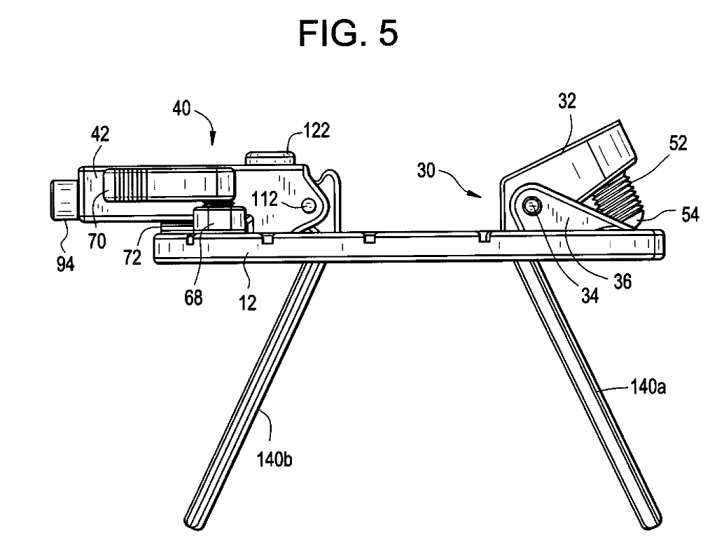

The access channel provided by the first and second retractor blades 30, 40 may be expanded by positioning the first retractor blade 20 and the second retractor blade 40 in a second, retraction configuration, in which the first retractor blade 20 and/or the second retractor blade 40 are adjusted away from one another. For example, one or both of the distal ends of the first retractor blade 20 and the second retractor blade 40 may be rotated away from one another and the second retractor blade 40 may be adjusted in a direction transverse to the axis of the curvate side 20 of the frame 12 away from the first retractor blade 30. FIGS. 4 and 5 illustrate a second, retracted configuration in which both of the distal ends of the first retractor blade 20 and the second retractor blade 40 are rotated away from one another and the second retractor blade 40 is adjusted in a direction transverse to the axis of the curvate side 20 of the frame 12 away from the first retractor blade 30. In addition, the length of the working channel may be increased by advancing an adjustable blade of one of the plurality of blades relative to a primary blade along a longitudinal axis of the primary blade, as illustrated in FIG. 3.

Additional retractor blades may be added to the first and second retractor blade 30, 40 and connected to the frame 12 to further retract tissue and further expand the access channel.

Any number of surgical procedures may be performed through the access channel including, for example, removal of some or all of one or more discs, placement of bone fusion promoting material, placement of an spine arthroplasty device such as an artificial disc, placement of spinal implants such as hooks, rods, and screws.

After the surgical procedure is performed, the retractor may be returned to the first, insertion configuration and removed from the incision.

While the devices and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

1. A retractor comprising:
a frame having a linear side having a first end and a second end and a curvate side connected at a first end to the first end of the linear side and at a second end to the second end of the linear side,
a first retractor blade connected to the linear side of the frame, and
a second retractor blade connected to the curvate side of the frame.

2. The retractor of claim 1, wherein the curvate side has an approximately constant radius along its length.

3. The retractor of claim 1, wherein the curvate side comprises a plurality of curvate segments having differing radii.

4. The retractor of claim 3, wherein the curvate side includes one or more linear segments.

5. The retractor of claim 1, wherein the first retractor blade is fixed to the linear side of the frame to inhibit movement of the first retractor blade along a longitudinal axis of the linear side and in a direction transverse to the longitudinal axis of the linear side.

6. The retractor of claim 5, wherein the first retractor blade is rotatable relative to the linear side.

7. The retractor of claim 6, further comprising a blade adjustment mechanism for selectively adjusting the rotational position of the first blade.

8. The retractor of claim 7, wherein the blade adjustment mechanism comprises a set screw engaged to a portion of the first retractor blade, the distal end of the set screw contacting the frame, rotation of the set screw about the axis of the set screw causing the. first blade to rotate about a rotation axis.

9. The retractor of claim 1, wherein the second retractor blade is adjustable relative to the curvate side of the frame in a direction transverse to an axis of the curvate side.

10. The retractor of claim 9, wherein the second retractor blade is rotatable relative to the curvate side.

11. The retractor of claim 1, wherein at least one of the first blade and the second blade comprises:
a primary blade, and
an adjustable blade operatively coupled to the primary blade and adjustable relative to the primary blade along a longitudinal axis of the primary blade.

12. The retractor of claim 1, wherein a segment of the frame is rotationally adjustable relative to another segment of the frame.

13. The surgical retractor of claim 1, wherein the curvate side of the frame is configured to receive one or more retractor blades along the length of the curvate side.

14. A retractor comprising:
a frame having a linear side having a first end and a second end and a curvate side connected at a first end to the first end of the linear side and at a second end to the second end of the linear side,
a first retractor blade connected to the linear side of the frame, the first retractor blade rotatable about the linear side, and
a second retractor blade connected to the curvate side of the frame, the second retractor blade rotatable about the curvate side, the retractor having an insertion configuration in which the distal end of the first retractor blade and the distal end of the second retractor blade are rotated into proximity to one another and a retracted configuration in which the distal end of the first retractor blade and the distal end of the second retractor blade are rotated away from one another.

15. The retractor of claim 14, wherein the first retractor blade is fixed to the linear side of the frame to inhibit movement of the first retractor blade along a longitudinal axis of the linear side and in a direction transverse to the longitudinal axis of the linear side.

16. The retractor of claim 15, wherein the second retractor blade is adjustable relative to the curvate side of the frame in a direction transverse to an axis of the curvate side.

17. A retractor comprising:
a frame having a linear side having a first end and a second end and a curvate side connected at a first end to the first end of the linear side and at a second end to the second end of the linear side,
a first retractor blade fixed to the linear side of the frame to inhibit movement of the first retractor blade along a longitudinal axis of the linear side and in a direction transverse to the longitudinal axis of the linear side, the first retractor blade rotatable about the linear side, and
a second retractor blade connected to the curvate side of the frame and adjustable relative to the curvate side of the frame in a direction transverse to an axis of the curvate side, the second retractor blade rotatable relative to the curvate side.

18. A method of providing minimally invasive access to spinal anatomy, the method comprising:
positioning a retractor in an insertion configuration by rotating a distal end of a first blade of a retractor into proximity to a distal end of a second blade of a retractor, the first blade of the retractor connected to a linear side of a frame of the retractor and the second blade connected to a curvate side of the frame of the retractor,
making an incision,
inserting the distal end of the first blade and the distal end of the second blade of the retractor through the incision with the retractor in the insertion configuration,
advancing the distal end of the first blade and the distal end of the second blade into proximity to the spinal anatomy with the retractor in the insertion configuration, and
adjusting at least one of the first blade and the second blade relative to the other blade to provide an access channel between the skin and the spinal anatomy.

19. The method of claim 18, further comprising positioning the linear side of the frame of the retractor medially with respect to the spine.

20. The method of claim 19, wherein the linear side of the frame is oriented parallel to the spine.

21. The method of claim 19, further comprising adjusting the first blade by rotating the first blade relative to the linear side of the frame.

22. The method of claim 19, further comprising adjusting the second blade in a direction transverse to the curvate side of the frame.

23. The method of claim 22, further comprising adjusting the second blade by rotating the second blade relative curvate side of the frame.

24. A retractor comprising:
a frame having a first segment that is rotationally adjustable relative to second segment of the frame,
a first retractor blade connectable to the first segment of the frame, and
a second retractor blade connectable to the second segment of the frame.

25. The retractor of claim 24, wherein the frame further comprises a third segment rotatably adjustable to at least one of the first segment and the second segment.

26. The retractor of claim 10, wherein the second blade is adjustable along the curvate side of the frame.
